(19) United States
(12) Patent Application Publication
(10) Pub. No.: US 2019/0183754 A1
(43) Pub. Date: Jun. 20, 2019

(54) High SPF, Photoprotective Anhydrous Zinc Oxide Sunscreen Compositions

(71) Applicant: Amavara, Inc., San Diego, CA (US)

(72) Inventor: Laura C. Singleton, Los Angeles, CA (US)

(21) Appl. No.: 16/220,353

(22) Filed: Dec. 14, 2018

Related U.S. Application Data
(60) Provisional application No. 62/598,837, filed on Dec. 14, 2017.

(51) Int. Cl. A61K 8/27; A61K 8/37; A61K 8/891; A61K 8/895; A61K 8/365; A61K 8/81; A61K 8/26; A61K 8/25; A61K 8/88; A61K 8/92; A61K 8/06; A61Q 17/04; A61Q 19/08

(52) U.S. Cl. CPC A61K 8/27; A61K 2800/5922; A61K 8/891; A61K 8/895; A61K 8/365; A61K 8/8141; A61K 8/26; A61K 8/25; A61K 8/88; A61K 8/922; A61K 8/064; A61Q 17/04; A61Q 19/08; A61K 2800/31; A61K 2800/41; A61K 8/37

(57) ABSTRACT

A broad spectrum, anhydrous sunscreen composition having an SPF value of at least 50 that (x) does not contain an organic sunscreen active that absorbs ultraviolet radiation and (y) contains only a physical sunscreen active ingredient, Zinc Oxide, having an average particle size of greater than about 100 nanometers, the composition being comprised of: (a) at least one SPF-boosting emollient selected from the group consisting of Octyldodecyl Neopentanoate, Butyl Octyl Salycilate, and Cetyl Dimethicone; (b) at least one powder that provides protection to the skin from photodamage induced by exposure to ultraviolet radiation; (c) at least one powder that provides protection of the skin from photodamage induced by exposure to infrared radiation; (d) at least one film-forming polymer, and at least one bulking agent or binding agent.

### FIELD OF INVENTION

Disclosed are anhydrous sunscreen compositions (i) having a “Sun Protection Factor” (SPF) value of at least 30, preferably at least 50 (see definition below), that are (ii) “broad spectrum” (according to regulations promulgated by both the US Food and Drug Administration (FDA) and the European Commission; see discussion below), (iii) do not contain an organic sunscreen active that absorbs ultraviolet radiation (UVR) and (iv) contains only a physical sunscreen active ingredient—“non-nano” Zinc Oxide. For purposes of the present application, a nanomaterial is a plurality of particles—in an unbound state, as an aggregate, or as an agglomerate—where, 50% or more of the particles having one or more external dimensions in the size range 1 nm-100 nm; a “non-nano” material does not meet the above definition.

### BACKGROUND OF THE INVENTION

In the United States, sunscreens are topically-applied products that (i) contain one or more sunscreen active ingredients, (ii) help prevent sunburn, and (iii), when classified as broad spectrum and having an SPF over 15 can, when used in accordance with directions, decrease the risks of skin cancer and early skin aging caused by the sun. Sunscreens that are not broad spectrum or that are broad spectrum with SPF values between 2 and 14 must be labeled in the United States with a warning “Skin Cancer/Skin Aging Alert”.

Sunscreen active ingredients are defined by the FDA as chemicals that absorbs, reflects, or scatters radiation in the UV range at wavelengths from 290 to 400 nanometers; and can be subdivided into “organic” sunscreens (that absorb UV radiation) and physical (that reflect or scatter UV radiation); the latter include Zinc Oxide and Titanium Dioxide.

Health concerns have been raised regarding certain “organic” sunscreen actives. For example, in June 2012, the California Environmental Protection Agency, Office of Environmental Health Hazard Assessment listed the organic sunscreen benzophenone as a chemical known to the State of California to cause cancer. See also report from the Environmental Working Group published at http://www.ewg.org/sunscreen/report/the-trouble-with-sunscreen-chemicals/, citing two publications (among others) by Janjua N R, et al.: Janjua N R, et al. “Systemic absorption of the sunscreens benzophenone-3, octyl-methoxycinnamate, and 3-(4-methyl-benzylidene) camphor after whole-body topical application and reproductive hormone levels in humans.” J Invest Dermatol. Vol. 123, pp. 57-61 (2004); Janjua N R, et al ” Sunscreens in human plasma and urine after repeated whole-body topical application. J Eur Acad Dermatol Venereol. Vol. 22, No. 4, pp. 456-461 (2008).

Additionally, health concerns have been raised with respect to the organic sunscreen octocrylene. In 2010, in an article entitled “Octocrylene, an emerging photoallergen” published in Archives of Dermatology [Vol. 146, No. 7, pp. at pages 753-757 (2010)], Avenel-Audran M et al. reported that octocrylene appears to be strong allergen leading to contact dermatitis in children and mostly photoallergic contact dermatitis in adults with an often-associated history of photoallergy from ketoprofen. See also, de Groot, AC and DW Roberts, “Contact and photocontact allergy to octocrylene: a review.” Contact Dermatitis, Vol. 70, pp. 193-204. (2014).

Historically, sunscreen formulations containing zinc oxide produced a white appearance caused by back-reflection of visible light from the zinc oxide particles. In response, scattering of light was minimized by reducing the size of the ZnO particles to the nanometer scale, (<100 nm). Additionally, improved transparency of ZnO particles was achieved by minimizing the difference in refractive indices of the particles and the excipient (e.g., inert carrier vehicle) in which the particles are dispersed.

However, potential health concerns have been raised about nanoparticles in sunscreens. See, for example, SE Cross et al. “Human Skin Penetration of Sunscreen Nanoparticles: In-vitro Assessment of a Novel Micronized Zinc Oxide Formulation” Skin Pharmacol. Physiol., Vol. 20, pp. 148-154 (2007).

Responsive to these concerns, transparent ZnO having average particle sizes larger than 100 nm were developed and marketed, including under the tradename ZinClear by Antaria Limited. See, US Pre-Grant Patent Application Publications 2010/0310871 and 2010/0316582, both abandoned.

Another example of ZnO marketed as having a particle size of greater than 100 nanometers is ARGA-SUN ZnO CLR-P. According to various Technical Data Sheets (TDS), ARGA-SUN ZnO CLR-P have a highly porous structure and an average particle size distribution of less than about 800 nanometers (measured using static laser scattering). Additionally, the pores of ARGA-SUN ZnO CLR-P particles are described as “infiltrated” (e.g., filled) with excipient, and having a refractive index close to the excipient, resulting in a “significant increase in transparency”.

SPF, the amount of solar energy (UV radiation) required to produce sunburn on protected skin (i.e., with sunscreen applied), is defined in terms of Minimal Erythema Dose (“MED”) according to the formula:

MED (protected skin)/MED (unprotected skin)

More particularly, SPF is the quantity of erythema-effective energy (expressed as Joules per square meter) required to produce the first perceptible, redness reaction with clearly defined borders. MED (protected skin) is the minimal erythemal dose for protected skin after application of 2 milligrams per square centimeter of a sunscreen product. MED (unprotected skin) is the minimal erythema dose for skin to which no sunscreen product has been applied.

“Critical Wavelength” is the wavelength for which the section under the integrated optical density curve starting at 290 nm is equal to 90 percent of the integrated section between 290 nm to 400 nm.

The final FDA Sunscreen Monograph—“Labeling and Effectiveness Testing; Sunscreen Drug Products for Over-the-Counter Human Use,” Vol. 76 Federal Register pp. 35620 (Jun. 17, 2011)—defines a “Broad Spectrum” Sunscreen as having Critical Wavelength of at least 370 nm and a SPF value of 15 or higher.

European Commission Recommendation of 22 Sep. 2006 (2006/647/EC) defines a “Broad Spectrum” Sunscreen as having (i) an SPF of more than 6, as determined in accordance with the International Sun Protection Factor Test Method of 2006 (or an equivalent in vitro method), (ii) UVA protection of more than ⅓ of the SPF as determined using the persistent pigment darkening method modified by the French health agency Agence française de sécurité sanitaire des produits de santé (or an equivalent in vitro method), and (iii) a critical wavelength of at least 370 nanometers.

Because the SPF of a sunscreen product is dependent, to a considerable extent, on the formulation, predicting how much sunscreen is need to achieve a given SPF is extremely difficult. http://www.crodapersonalcare.com/home.aspx?s=157&r=265&p=2046. For example, Croda explains “For zinc oxide, 1% solids should give about 1 SPF unit. [Z]inc oxide dispersions contain 60% solids, so a concentration of 10% dispersion in the formulation should give an SPF of about 6.” See, A. Nayyir-Mazhir & K. McNeil, “Formulating broad spectrum UV protection products” Personal Care, pp. 21-24 (November 2013); see also, https://www.makingcosmetics.com/How-Can-I-Get-a-SPF_ep_60. html (accessed on DATE). Technical data sheets from some suppliers of specialty chemicals teach that 1% of ZnO in a formulation can contribute up to about 1.5 units of SPF. See, Technical Data Sheet from EverZinc for Zano® 10, Zano® 20 & Zano® M (Nov. 25, 2016).

The photostability of a UV filter can be measured and expressed in terms of the amount (i.e., concentration) by which the filter is degraded by one or more photochemical reactions, including photofragmentation, isomerization, tautomerization, photoaddition and substitution reactions, and/or cycloadditions. These photodegradation reactions also generate free radicals, which are associated with adverse health consequences.

Sunscreens are considered “photostable” if the area under a spectral absorption curve after irradiation with a solar simulator is 80% of the area under the spectral absorption curve prior to irradiation.

Increasingly, consumers are demanding photostable sunscreen products that provide broad spectrum protection and have a high SPF, preferably of at least 50. Achieving this level of sun protection without using organic sunscreen filters in an aesthetically elegant formulation—one that is non-whitening or oily when applied to skin—are needs have, and continue, to exist; and are met by compositions of the present invention.

### DETAILED DISCUSSION OF THE INVENTION

The present invention is directed to anhydrous sunscreen compositions
(i) having an SPF of at least 50, that are
(ii) “broad spectrum” (according to regulations promulgated by both the US Food and Drug Administration (FDA) and the European Commission)
(iii) photostable
(iv) do not contain an organic sunscreen active that absorbs ultraviolet radiation, and
(v) contain only a single physical sunscreen active ingredient Zinc Oxide having an average particle size of greater than about 0.1 microns.

### Zinc Oxide

Sunscreen compositions of the present invention contain a single sunscreen active ingredient—“Non-Nano Zinc Oxide”—uncoated particles of Zinc Oxide having an average particle size of greater than about 0.1 microns, preferably having an average particle size of greater than about 0.2 microns, still more preferably having an average particle size of greater than about 0.4 microns, and even more preferably having an average particle size of greater than about 0.5 microns and less than about 0.8 microns.

In preferred embodiments, the Non-Nano Zinc Oxide particles have a porous structure, such that when the pores are filled with an excipient (carrier fluid), the refractive index of the particles is reduced to a value close to that of the excipient. Non-Nano Zinc Oxide particles are commercially available from Argan Co. under the tradename ARGA-SUN ZnO CLR-P.

### SPF-Boosting Emollients

Emollients are cosmetic ingredients that help to maintain the soft, smooth appearance of skin and reduce flaking.

Compositions of the present invention include at least one, preferably at least two, and even more preferably at least three emollients that boost (i.e., increase) SPF, preferably selected from the group consisting of Octyldodecyl Neopentanoate, Butyl Octyl Salycilate, and Cetyl Dimethicone.

Octyldodecyl Neopentanoate (C₂₅H₅₀O₂) is sold under the tradename Elefac I-205 by Alzo International Inc. (Sayreville, N.J.) is Octyldodecyl Neopentanoate. It is the ester of octyldodecanol (q.v.) and neopentanoic acid and conforms to the formula:

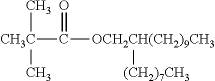

Butyloctyl Salicylate, commercially available as HallBrite® BHB from the Hallstar Company is a synthetically produced ester of Salicylic Acid and a branched C₁₂ alcohol, 2-butyloctanol. Butyloctyl Salycilate has an empirical formula of C₁₉H₃₀O₃ and the following structural formula:

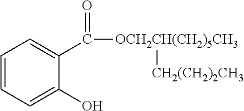

Use of Butyloctyl Salicylate in sunscreen products is described in the following U.S. Pat. Nos. 5,783,173; 5,788,954; 5,849,273; and 6,350,894.

Cetyl Dimethicone is a dimethyl siloxane polymer that conforms to the formula:

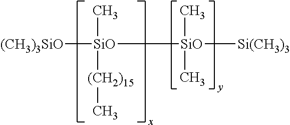

It is commercially available from a number of specialty chemical companies, including under the tradename ABIL® Wax 9801 is commercially available from Evonik Goldschmidt GmbH (Essen, Germany).

Compositions of the present invention may, and preferably do, include additional emollients, including, but not limited to Dimethicone and Caprylic/Capric Triglyceride.

Dimethicone is a mixture of fully methylated linear siloxane polymers end blocked with trimethylsiloxy units, that conforms to the formula:

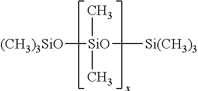

Caprylic/Capric Triglyceride is the mixed triester of glycerin and caprylic and capric acids.

### Photodamage Reducing Ingredients (Ultraviolet and Infrared Radiation)

Photodamage can result from unprotected skin exposure to ultraviolet radiation (UVR) and infrared radiation (IR). Compositions of the present invention include (i) at least one particle (also referred to below as a powder) that increases protection of the skin from UVR-induced photodamage (a “UVR-Protective Powder”) and (ii) at least one particle that blocks, and therefore, provides protection from IR-induced photodamage (an “IR-Protective Powder”). In one aspect of the present invention, by the phrase “increases protection of the skin from UVR-induced photodamage” means increases SPF.

In paint embodiments of the present invention, one preferred UVR-Protective Powder is Styrene/Acrylates Copolymer, sold under the tradename Sunspheres from Dow. This polymer of styrene and a monomer consisting of acrylic acid, methacrylic acid or one of their simple esters is manufactured via emulsion polymerization creating what technical literature from Dow describes as “hollow spheres . . . that raise the UV protection over the whole UVA/UVB spectrum and work equally well with organic and inorganic sunscreen actives . . . and is also compatible with a broad range of cosmetic ingredients, including emollients and skin conditioners.”

The technical brochure from Dow continues to state generally, “The ability of SUNSPHERES to boost the efficacy of UVA/UVB filters of formulations allows the formulator to use significantly less active to deliver the same level of SPF.”

Another preferred UVR-Protective Ingredient is Polyhydroxystearic Acid—a polymer of Hydroxystearic Acid (q.v.) that is commercially available under the tradename Dispersun DSP OL 300 from Innospec Performance Chemicals (Salisbury, N.C.).

According to technical literature from the specialty chemical company Innospec, Polyhydroxystearic Acid increases UV absorption of sunscreens containing pigments, including by allowing higher concentrations of pigment to be used. Additionally, inclusion of Polyhydroxystearic Acid is described by Innospec as improving optical transparency and reduce whitening (when finished formulation is applied to skin).

One preferred IR-Protective Particle is ARG-SPHERE NIR-1/15BA000—a combination of PMMA, Cerium Oxide, and Aluminum Oxide from Argan Co.

Poly(methyl methacrylate), also known in the art as PMMA, is a spherical ultra-fine texturizing powder, available in various sizes, used in powders to increase smoothness, fluidity and lubricity. More particularly, PMMA is a polymer of methyl methacrylate, has an empirical formula C₅H₈O₂)ₓ and conforms to the following structure:

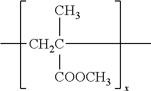

Cerium Oxide (CeO) and Aluminum Oxide (Al₂O₃) are inorganic oxide compounds.

### Aesthetic Enhancing Powders

Compositions of the present invention preferably include one powder that enhances skin feel, more preferably more than one powder. In preferred embodiments, the powder reduces the feeling of oiliness that is commonly associated with sunscreen formulations. Ingredients that provide skin-feel improving benefits are referred to in the present application as “Aesthetic Enhancing Powders”.

One Aesthetic Enhancing Powder that is preferably included in compositions of the present invention is “oil-absorbing”, by which is meant the powder has an internal porosity sufficient to absorb oils and esters and, thereby, contribute to a non-greasy skin-feel.

In one preferred embodiment, the oil-absorbing powder has an absorption capacity of at least about 20 ml/100 g as measured by the test method in US Pre-Grant Patent Application 2011/0293543 ¶ [0036]. In another preferred embodiment, oil absorption capacity is determined by weighing approximately 2 to 2.2 g of the powder (sieved through 200 mesh screen in advance) and adding liquid paraffin until loose and dry powder disappears, and expressed according to the following formula: final volume of liquid paraffin absorbed (in cm³)/weight of sample (in grams).

One particularly preferred oil absorbing Aesthetic Enhancing Powder is silica, commercially available from a number of speciality chemical suppliers, including under the tradename Silisphere LS-8H from Argan Co.

In addition, the oil-absorbing powder contributes to homogeneity (i.e., even dispersion of pigments), and also helps to provide soft focus and skin mattifying effects.

The oil-absorbing powder also provides additional processing/manufacturing benefits, including reducing agglomeration (“clumping”).

In certain preferred embodiments, the compositions of the present invention include, as an Aesthetic Enhancing Powder, Nylon 12 microbeads coated with a film of PMMA and available from Argan Co. as ARG-PMMA-10NYL.

Nylon-12 is known in the art to impart elegant silky texture to finished products, enhance slip, increase blendability, and reduce the appearance of fine lines and wrinkles on the skin. More particularly, Nylon-12 is a spherical, polymeric powder used in cosmetic applications; it is a polyamide derived from 12-aminododecanoic acid and conforms generally to the formula:

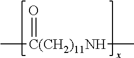

### Emulsifier

Compositions of the present invention are water-in-oil (“W/O”) emulsions. By the term W/O emulsion is to be understood a two-phase mixture of immiscible liquids—an inner phase containing water-soluble (hydrophilic) ingredients dispersed in a continuous outer phase on hydrophobic ingredients (oils, esters, and silicones).

A preferred W/O emulsifier is Polyglyceryl-3 Sorbityl Linseedate is a water-in-silicone emulsifier sold by Sinerga/Argan Co. under the tradename EWOCREAM. More Polyglyceryl-3 Sorbityl Linseedate is the product obtained by the reaction of Linseed Acid (q.v.) with the sorbitol ether of Polyglycerin-3 (q.v.).

### Film-Forming Polymer

Compositions of the present invention contain at least one film-forming polymer that imparts water repellency and wash-off resistance.

One preferred film-forming polymer is Acrylates/Dimethicone Copolymer—a copolymer of dimethicone and one or more monomers of acrylic acid, methacrylic acid or one of their simple esters—available from numerous suppliers, including Access Ingredients (South Pasadena, Calif.) under the tradename AccessSIL FF-16.

Another polymer that provides transfer resistance (of colored pigments) as well as repellency of water and sebum, is a mixture of trimethylsiloxysilicate and isododecane, available under the tradename JEESILC® TMR-40.

Trimethylsiloxysilicate is the siloxane polymer that conforms generally to the formula:

[(CH₃)₃SiO_{1/2}]ₓ[SiO₂]_{y}

Isododecane is a branched chain aliphatic hydrocarbon with 12 carbons.

### Bulking and Binding Agents

Bulking agents are chemically inert, solid ingredients employed as diluents for other solids. Bulking agents can also serve as “extenders” of pigments.

Binding agents are ingredients added to compounded dry powder mixtures to provide adhesive qualities during and after compression.

Compositions of the present invention include one or more bulking agents, preferably selected from the group consisting of Hectorite (and its derivatives and reaction products), Kaolin and Polyethylene.

Hectorite is one of the montmorillonite minerals that are the principal constituents of bentonite clay.

Kaolin is a native hydrated aluminum silicate having the structure Al₂O₃.2SiO₂.2H₂O.

Disteardimonium Hectorite is the reaction product Hectorite and Distearyldimonium Chloride, a quaternary ammonium salt that conforms generally to the formula:

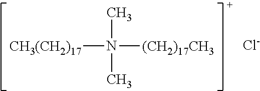

Disteardimonium Hectorite is commercially available from Elementis Specialities, Inc. (Hightstown, N.J.) under the tradename Bentone 38V. A technical data sheet from Elementis describes Bentone 38 as a rheological additive for low to intermediate polarity organic systems, that increases viscosity, provides thixotropy while preventing synerisis, and prevents pigment settling during storage.

Polyethylene is a polymer of ethylene monomers conforming to the formula:

—CH₂CH₂ₓ

and is available in a number of forms/grades from a variety of suppliers to the personal care industry, including JEEN International under the tradenames JEENATE 3H and 5H.

One preferred binding agent is Ozokerite, a hydrocarbon wax derived from mineral or petroleum sources, and available from a number of suppliers of chemical raw materials to the personal care industry. A particularly preferred Ozokerite is sold by Koster Keunen under the tradename Ozokerite 2095, a high melt point wax blend consisting of branched and linear hydrocarbons—namely, Microcrystalline Wax and Paraffin.

Another preferred binding agent is Polybutene, formed by the polymerization of a mixture of iso- and normal butenes.

### Skin Conditioning Agents

Compositions of the present invention include at least one, preferably a plurality of skin conditioning agents—ingredients that enhance the appearance of dry or damaged skin. Without wishing to be bound by a specific mechanism of action, conditioning agents adhere to the skin to reduce flaking and restore suppleness.

One non-limiting example of a skin conditioning agent that may be, and preferably is, included in compositions of the present invention is Helianthus Annus (Sunflower) Seed Wax, an article of commerce available from numerous chemical raw materials suppliers including JEEN International under the tradename JeeWax Sun.

Preferably at least one of the skin conditioning agents is occlusive, by which is meant the ingredient retards evaporation of water from the skin surface (i.e., transepidermal water loss); by blocking the evaporative loss of water, occlusive skin conditioning agents increase the water content of skin.

One preferred occlusive skin conditioning agent is Tribehenin (C₆₉H₁₃₄O₆), the triester of glycerin and behenic acid, conforms to the formula:

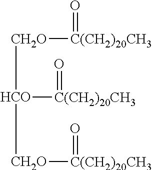

and is commercially available under the tradename Syncrowax HR-C from Croda Inc.

Another preferred occlusive skin conditioning agent is a mixture of Hydrogenated Olive Oil and Hydrogenated Olive Oil Unsaponifiables, that is commercially available from Sinerga/Argan Co. under the tradename Wax OLEA.

### Aesthetics Modifier

As Aesthetics Modifiers, compositions of the present invention can, and preferably do contain, Nonaqueous Viscosity Increasing Agents—namely, ingredient used to thicken or gel the water-insoluble portions of cosmetic products.

One preferred Aesthetics Modifier is Dimethicone/Vinyl Dimethicone Crosspolymer, a crosslinked dimethyl siloxane polymer (silicone elastomer) that is formed by the reaction between Hydrogen Dimethicone (q.v.) or Bis-Hydrogen Dimethicone (q.v.), and Vinyl Dimethicone (q.v.) or Bis-Vinyldimethicone (q.v.).

Hydrogen Dimethicone is a derivative of Dimethicone (q.v.) where some of the methyl groups have been replaced with a hydrogen atom. It conforms generally to the formula:

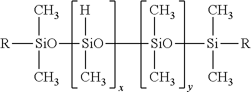

where R can be either hydrogen or a methyl group.

Bis-Hydrogen Dimethicone is the siloxane compound that conforms to the formula:

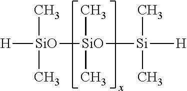

Vinyl Dimethicone is a derivative of Dimethicone (q.v.) where some of the methyl groups have been replaced with vinyl groups. The vinyl groups can occur at the ends of the siloxane chain or pendant to the siloxane chain. It conforms generally to the formula:

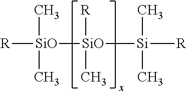

where R is a methyl or vinyl group, and at least one vinyl group is present.

Bis-Vinyldimethicone is a derivative of Dimethicone (q.v.) where one methyl group at each end of the siloxane chain has been replaced with a vinyl group. It conforms generally to the formula:

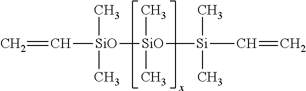

Emul-6081 from Access Ingredients is a mixture of Dimethicone/Vinyldimethicone Crosspolymer (and) Laureth-3 (and) Laureth-25.

### Environmental Protectants

Compositions of the present invention include one or more Environmental Protectants selected from antioxidants/free-radical quenchers and anti-irritants/anti-inflammatory agents. Three particularly preferred Environmental Protectants are

Benzylidene Dimethoxydimethylindanone (C₂₀H₂₀O₃) is an organic compound that conforms to the formula:

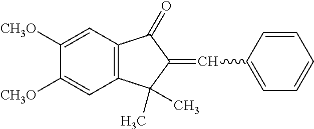

and is commercially available under the tradename SymUrban® from Symrise. SymUrban® is marketed as an “anti-pollution agent” that reduces environmental damage—namely, collagen degradation, overproduction of melanin and inflammation resulting from exposure to airborne particulate matter found in vehicle exhaust and industrial emissions.

Tocopheryl Acetate is the ester of Tocopherol (q.v.) and acetic acid, and is an article of commerce supplied to the personal care industry by a several companies.

A mixture of Bisabolol and Zingiber Officinale (Ginger) Root Extract is commercially available under the tradename SymRelief 100 from Symrise.

Bisabolol is a terpene that conforms to the formula:

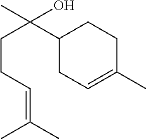 [SMILES: CC(C)=CCCC(C)(O)C1CC=C(C)CC1]

### Preservative System

Compositions of the present invention do not use parabens or formaldehyde-releasing ingredients.

In preferred embodiments, compositions of the present invention include Hydroxyacetophenone, 1,2 Hexanediol and Caprylyl Glycol.

Hydroxyacetophenone (C₈H₈O₂) available under the tradename Symsave H from Symrise is the organic compound that conforms to the formula:

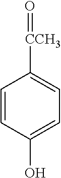

A mixture of 1,2 Hexanediol and Caprylyl Glycol is commercially available under the tradename SymDiol 68 from Symrise.

### Skin Coloring Agents

Compositions of the present invention may, and preferably do contain, as a skin protecting agent, one or more Iron Oxide pigment(s) and/or other natural colorants (e.g., Caramel).

When present, Iron Oxide pigments may be, and preferably are, coated.

One preferred coating is Hydrogenated Lecithin, the end-product of the controlled hydrogenation of Lecithin (q.v.).

### Formulation Example 1—Untinted Stick (SPF 50+)

| INCI Name | Range % |
|---|---|
| Octyldodecyl Neopentanoate | 10.00-20.00 |
| Caprylic/Capric Triglyceride | 5.00-15.00 |
| Butyl Octyl Salycilate | 15.00-25.00 |
| Polyhydroxystearic Acid | 0.10-2.00 |
| Dimethicone 50 cst | 1.00-5.00 |
| Disteardimonium Hectorite | 0.10-2.00 |
| Helianthus Annus Seed Wax | 0.10-1.00 |
| Ozokerite | 3.00-7.00 |
| Polyethylene | 1.00-5.00 |
| Tribehenin | 0.10-1.00 |
| Hydrogenated Olive Oil (and) Hydrogenated Olive Oil | 3.00-7.00 |
| Unsaponifiables | |
| Bisabolol and Zingiber Officinale (Ginger) Root Extract | 0.10-1.00 |
| Dimethicone (and) Acrylates/Dimethicone Copolymer | 0.10-3.00 |
| Dimethicone/Vinyldimethicone Crosspolymer (and) | 0.10-3.00 |
| Laureth-3 (and) Laureth-25 | |
| Tocopheryl Acetate | 0.10-1.00 |
| 1,2 Hexanediol and Caprylyl Glycol | 0.10-1.00 |
| Benzylidene Dimethoxydimethylindanone | 0.01-0.50 |
| Zinc Oxide (non-nano) | 20.00-25.00 |
| Phenethyl Alcohol and Caprylyl Glycol | 0.10-1.00 |
| Silica | 3.00-7.00 |
| PMMA and Nylon 12 | 2.00-5.00 |
| PMMA, Cerium Oxide, Aluminum Oxide | 0.10-2.00 |

### Formulation Example 2—Untinted Compact/Pressed Powder (SPF 50+)

| INCI Name | Range % |
|---|---|
| Octyldodecyl Neopentanoate | 10.00-20.00 |
| Caprylic/Capric Triglyceride | 3.00-7.00 |
| Butyl Octyl Salycilate | 15.00-25.00 |
| Polyhydroxystearic Acid | 0.10-2.00 |
| Dimethicone 50 cst | 1.00-5.00 |
| Disteardimonium Hectorite | 0.10-2.00 |
| Helianthus Annus Seed Wax | 0.10-1.00 |
| Ozokerite | 3.00-7.00 |
| Polyethylene | 0.10-3.00 |
| Tribehenin | 0.10-3.00 |
| Hydrogenated Olive Oil (and) Hydrogenated Olive Oil | 3.00-7.00 |
| Unsaponifiables | |
| Bisabolol and Zingiber Officinale (Ginger) Root Extract | 0.10-1.00 |
| Dimethicone (and) Acrylates/Dimethicone Copolymer | 0.10-3.00 |
| Dimethicone/Vinyldimethicone Crosspolymer (and) | 0.10-3.00 |
| Laureth-3 (and) Laureth-25 | |
| Tocopheryl Acetate | 0.10-1.00 |
| 1,2 Hexanediol and Caprylyl Glycol | 0.10-1.00 |
| Benzylidene Dimethoxydimethylindanone | 0.01-0.50 |
| Zinc Oxide (non-nano) | 20.00-25.00 |
| Phenethyl Alcohol and Caprylyl Glycol | 0.10-1.00 |
| Silica | 3.00-7.00 |
| PMMA and Nylon 12 | 3.00-7.00 |
| PMMA, Cerium Oxide, Aluminum Oxide | 0.10-2.00 |

### Formulation Example 3—Face/Body Paint

| INCI Name | Range % |
|---|---|
| Butyl Octyl Salycilate | 15.00-25.00 |
| Caprylic/Capric Triglyceride | 4.00-8.00 |
| Polyhydroxystearic Acid | 0.10-2.00 |
| Styrene/Acrylates Copolymer | 1.00-5.00 |
| Benzylidene Dimethoxydimethylindanone | 0.01-0.50 |
| Tocopheryl Acetate | 0.10-1.00 |
| 1,2 Hexanediol and Caprylyl Glycol | 0.10-1.00 |
| Zinc Oxide (non-nano) | 20.00-25.00 |
| Kaolin | 12.00-20.00 |
| Polyglyceryl-3 Sorbityl Linseedate | 0.10-1.00 |
| Iron Oxides and Hydrogenated Lecithin | 1.00-5.00 |
| Titanium Dioxides and Hydrogenated Lecithin | 2.00-6.00 |
| Iron Oxides and Hydrogenated Lecithin | 0.01-1.00 |
| Iron Oxides and Hydrogenated Lecithin | 0.01-1.00 |
| Helianthus Annus (Sunflower) Seed Wax | 0.10-1.00 |
| Ozokerite | 1.00-5.00 |
| Polyethylene | 1.00-5.00 |
| Hydrogenated Olive Oil and Hydrogenated Olive Oil | 0.10-1.00 |
| Unsaponifiables | |
| Disteardimonium Hectorite | 0.01-1.00 |
| Polybutene | 1.00-5.00 |
| Isododecane and Trimethylsiloxysilicate | 4.00-8.00 |
| Bisabolol and Zingiber Officinale Root Extract | 0.10-1.00 |
| PMMA, Cerium Oxide, Aluminum Oxide | 0.10-2.00 |
| Polymethylsilsesesquioxane and Titanium Dioxide | 0.10-5.00 |

1. A broad spectrum, anhydrous sunscreen composition having an SPF value of at least 50 that (x) does not contain an organic sunscreen active that absorbs ultraviolet radiation and (y) contains only a physical sunscreen active ingredient, Zinc Oxide, having an average particle size of greater than about 100 nanometers, the composition being comprised of:
a. at least one SPF-boosting emollient selected from the group consisting of Octyldodecyl Neopentanoate, Butyl Octyl Salycilate, and Cetyl Dimethicone,
b. at least one powder that provides protection to the skin from photodamage induced by exposure to ultraviolet radiation,
c. at least one powder that provides protection of the skin from photodamage induced by exposure to infrared radiation,
d. at least one film-forming polymer, and
e. at least one bulking agent or binding agent.

2. The sunscreen composition of claim 1, wherein the at least one powder that provides protection to the skin from photodamage induced by exposure to ultraviolet radiation is selected from the group of Styrene/Acrylates Copolymer or Polyhydroxystearic Acid.

3. The sunscreen composition of claim 1, wherein the at least one powder that provides protection of the skin from photodamage induced by exposure to infrared radiation is a mixture that contains Cerium Oxide and Aluminum Oxide.

4. The sunscreen composition of claim 1, further comprising at least one aesthetic enhancing powder that improves skin feel, is oil-absorbing, imparts silky texture, enhances slip, increases blendability, or reduces the appearance of fine lines and wrinkles on skin.

5. The sunscreen composition of claim 1, wherein the aesthetic enhancing powder is oil-absorbing.

6. The sunscreen composition of claim 5, wherein the oil-absorbing aesthetic enhancing powder is silica.

7. The sunscreen composition of claim 1, wherein the aesthetic enhancing powder is Nylon-12.

8. The sunscreen composition of claim 1, wherein the film-forming polymer is selected from the group consisting of (a) Acrylates/Dimethicone Copolymer and (b) Trimethylsiloxysilicate.

9. The sunscreen composition of claim 1, further comprising at least one bulking agent selected from the group consisting of Hectorite (and its reaction products), Kaolin and Polyethylene and at least one binding agent selected from the group of Ozokerite and Polybutene.

10. The sunscreen composition of claim 1, further comprising a water-in-oil emulsifier.

11. The sunscreen composition of claim 10, wherein the water-in-oil emulsifier is Polyglyceryl-3 Sorbityl Linseedate.

12. (canceled)

13. (canceled)

14. The sunscreen composition of claim 13, wherein the at least one occlusive skin conditioning agent is Tribehenin or a mixture of Hydrogenated Olive Oil and Hydrogenated Olive Oil Unsaponifiables.

15. The sunscreen composition of claim 1, further comprising an aesthetics modifier.

16. The sunscreen composition of claim 15, wherein the aesthetics modifier is a nonaqueous viscosity increasing agent.

17. The sunscreen composition of claim 16, wherein the nonaqueous viscosity increasing agent is Dimethicone/Vinyl Dimethicone Crosspolymer.

18. The sunscreen composition of claim 1, further comprising at least one environmental protectant.

19. The sunscreen composition of claim 18, wherein the at least one environmental protectant is selected from the group of Benzylidene Dimethoxydimethylindanone, Tocopheryl Acetate, Bisabolol and Zingiber Officinale Root Extract.

20. The sunscreen composition of claim 1, further comprising at least one coloring agent.

21. (canceled)

22. (canceled)

23. (canceled)

24. (canceled)

25. The sunscreen composition of claim 1, wherein the Zinc Oxide has a porous structure, such that when pores within Zinc Oxide particles are filled with an excipient, the refractive index of the Zinc Oxide particles is reduced.

26. The sunscreen composition of claim 1 having water resistance for 80 minutes as determined in accordance with the test method codified at 21 CFR § 201.327(i)(7).
